(19) United States
(12) Patent Application Publication
(10) Pub. No.: US 2004/0116800 A1
(43) Pub. Date: Jun. 17, 2004

(54) Magnetic resonance imaging capable catheter assembly

(76) Inventors: Jeffrey L. Helfer, Webster, NY (US); Robert W. Gray, Rochester, NY (US); Michael L. Weiner, Webster, NY (US)

Correspondence Address: HOWARD J. GREENWALD P.C., 349 W. COMMERCIAL STREET SUITE 2490, EAST ROCHESTER, NY, 14445-2408, US

(21) Appl. No.: 10/369,429

(22) Filed: Feb. 19, 2003

Related U.S. Application Data
(60) Provisional application No. 60/357,935, filed on Feb. 19, 2002.

(51) Int. Cl. A61B 5/05

(52) U.S. Cl. 600/411

(57) ABSTRACT

A catheter assembly which is provided with a distally positioned magnetic resonance imaging coil, comprising a cable assembly having a proximal end and a distal end, the cable assembly further comprising an outer tube, a first electronics assembly disposed within the distal end of the cable assembly, a first fiber optic strand disposed within the tube, and connected to the first electronic assembly; and a tip assembly connected to the distal end of the cable assembly further comprising a thin structural wall forming a cavity, and a coil assembly disposed within the cavity. The catheter assembly enables high resolution magnetic resonance imaging of tissue proximate to the assembly, as well as other beneficial diagnostic and therapeutic procedures.

### CROSS-REFERENCE TO RELATED PATENT APPLICATIONS

This application claims the benefit of the filing date of U.S. provisional patent application Serial No. 60/357,935 filed Feb. 19, 2002.

This invention relates in one embodiment to a catheter assembly, and more particularly to a catheter assembly that includes the capability to perform magnetic resonance imaging.

### FIELD OF THE INVENTION

A catheter assembly which is provided with a distally positioned magnetic resonance imaging coil, thereby enabling high resolution magnetic resonance imaging of tissue proximate to the assembly.

### BACKGROUND OF THE INVENTION

Magnetic resonance imaging (MRI) is rapidly becoming an imaging method of choice for most non-invasive diagnostic procedures due to a variety of advantages. MRI is particularly effective in the imaging of internal organs, because images produced by MRI have superb soft tissue contrast, the imaging process is not obstructed by bone, and it is straightforward to obtain multi-plane images without repositioning patient. MRI is harmless to a majority of patients, as it requires no ionizing radiation or toxic contrast agents. It provides highly precise and clear images, thereby enabling functional analysis capabilities and a rapidly emerging medical practice of MRI-guided surgery.

However there remains opportunity for further improvement of MRI. Present MRI capabilities are still unable to image disease conditions where exceptional tissue morphological or spectral resolution is required, such as the diagnosis of “vulnerable plaques” (see Peter Libby, “Atherosclerosis: The New View,” Scientific American, May 2002, Volume 286, number 5, pages 46-55). It is well known to those skilled in the art that reducing the distance between the tissues to be imaged by MRI and the receive coil in the MRI unit will enhance the signal from the tissues and thereby improve the quality of the magnetic resonance image, specifically by improving the tissue magnetic resonance image signal-to noise ratio.

The present invention provides such a reduction in the distance between the tissues to be imaged by MRI. The present invention provides a small diameter MRI imaging coil that can be placed within the body, such as natural body openings or punctures through the skin, and to enable the coil to be positioned close to the tissues to be imaged, thereby providing significant improvement in morphological or spectral image quality due to the enhanced signal from the tissues and the increase in tissue magnetic resonance image signal-to-noise ratio that this closer proximity provides. The present invention may be further combined with other diagnostic and therapeutic features and capabilities useful for the diagnosis and treatment of diseases. In the preferred embodiment, the present invention is provided as a catheter device.

Heretofore, a number of patents and publications have disclosed catheter devices, the relevant portions of which may be briefly summarized as follows:

U.S. Pat. No. 6,236,879, for a “Fiber optic catheter system,” discloses “A catheter system including a catheter having a proximal end and a distal end and a device for determining the position of the distal end of the catheter relative to the position of the proximal end of the catheter, the device for determining the position including a glass fiber within a lumen of the catheter, the lumen being defined by a wall, a first polarization filter near the proximal end of the catheter, and a second polarization filter near the distal end of the catheter, wherein the first and second polarization filters are fixed with respect to the wall, and wherein the glass fiber is suitable for transporting polarized light while maintaining the direction of the polarization of the light substantially unchanged during torsional stress of the catheter.”

U.S. Pat. No. 6,166,806, for a “Fiber optic catheter for accurate flow measurements,” discloses “A two-fiber optic probe or sensor performs accurate measurements of fluids flowing within a remote vessels, such as blood flowing within arteries or veins or fluid flowing within pipes.”

U.S. Pat. No. 5,973,779, for a “Fiber-optic imaging probe,” discloses “A fiber-optic imaging probe is disclosed for use in dynamic light scattering applications. The probe includes two monomode optical fibers and two GRIN lenses to form a pair of identical fiber-lens combinations.”

U.S. Pat. No. 5,415,653, for a “Optical catheter with stranded fibers,” discloses “A catheter having an axis extending between a proximal end and an opposing distal end includes a plurality of optical fibers arranged to spiral in a first direction to form a circumferential layer around the axis.”

U.S. Pat. No. 4,991,590, for a “Fiber optic intravascular blood pressure transducer,” discloses “A device for the measurement of the blood pressure of a patient includes an arrangement for transmitting a light through an optical fiber; an arrangement for receiving and measuring a reflected light through an optical fiber; and a cylindrically shaped pressure sensor having a side window and a plate having two sections which moves in accordance with the applied blood pressure thereby causing the reflection and detection of different amounts of light based on the applied blood pressure at the window.”

U.S. Pat. No. 5,919,135, for a “System and method for treating cellular disorders in a living being,” discloses “ . . . . The invention employs a computerized imaging system (such as CAT scan, MRI imaging, ultrasound imaging, infrared, X-ray, UV/visible light fluorescence, Raman spectroscopy, single photon emission computed tomography or microwave imaging) to sense the position of a drug infusing catheter within the body . . . . ”

U.S. Pat. No. 6,026,316, for a “Method and apparatus for use with MR imaging,” discloses, “The invention is an apparatus and method for targeted drug delivery into a living patient using magnetic resonance (MR) imaging. The apparatus and method are useful in delivery to all types of living tissue and uses MR Imaging to track the location of drug delivery and estimating the rate of drug delivery. An MR-visible drug delivery device positioned at a target site (e.g., intracranial delivery) delivers a diagnostic or therapeutic drug solution into the tissue (e.g., the brain). The spatial distribution kinetics of the injected or infused drug agent are monitored quantitatively and non-invasively using water proton directional diffusion MR imaging to establish the efficacy of drug delivery at a targeted location.”

U.S. Pat. No. 6,052,613, “Blood pressure transducer,” discloses, “This invention relates to a blood pressure transducer (8) and provides a safe and economical transducer by providing a novel optical fiber (80) made of a transparent elastomer. The present invention provides an invasive direct blood pressure transducer (8) of an external sensor system consisting of a catheter (1a), a pressure tub (6) connected to the catheter at one of the ends thereof and a pressure transducer (8) connected to the other end of the pressure tube (6), part of the pressure transducer is composed of an optical fiber (80) made of a transparent elastomer.”

U.S. Pat. No. 5,445,151, for a “Method for blood flow acceleration and velocity measurement using MR catheters,” discloses “A method of magnetic resonance (MR) fluid flow measurement within a subject employs an invasive device with an RF transmit/receive coil and an RF transmit coil spaced a known distance apart. The subject is positioned in a static magnetic field. The invasive device is positioned in a vessel of a subject in which fluid flow is desired to be determined. A regular pattern of RF transmission pulses are radiated through the RF transmit/receive coil causing it to cause a steady-state MR response signal. Intermittently a second RF signal is transmitted from the RF coil positioned upstream, which causes a change in the steady-state MR response signal sensed by the downstream transmit/receive coil. This is detected a short delay time later at the RF receive coil. The time delay and the distance between the RF coils lead directly to a fluid velocity. By exchanging the position of the RF transmit and transmit/receive coils, retrograde velocity may be measured. In another embodiment, more RF coils are employed. The changed MR response signal may be sensed at a number of locations at different times, leading to a measured change in velocity, or acceleration of the fluid.”

U.S. Pat. No. 6,134,003, for a “Method and apparatus for performing optical measurements using a fiber optic imaging guidewire, catheter or endoscope,” discloses, “An imaging system for performing optical coherence tomography includes an optical radiation source; a reference optical reflector; a first optical path leading to the reference optical reflector; and a second optical path coupled to an endoscopic unit.”

U.S. Pat. No. 5,830,209, for a “Multi-fiber laser catheter,” discloses “Laser catheters according to the invention include multiple optical fibers for delivery of laser energy to a pre-determined treatment site in the therapeutic treatment of cardiac tissue. A fixation device fixes the distal end of the catheter to the treatment site. Temperature sensing devices disposed on the fixation device provide a temperature depth profile of the tissue treatment site, which can be used to control the treatment. Multi-piece, single-piece and porous tip catheters are disclosed.”

U.S. Pat. No. 6,024,738, for a “Laser catheter apparatus for use in arteries or other narrow paths within living organisms,” discloses “A laser catheter for the treatment of lesions and plaque deposits in arteries and other narrow paths having a radiation assembly affixed to a flexible conduit. The conduit generally includes multiple lumens for the passage of an optical fiber, a guide wire, a cooling medium therethrough, or fluid for inflating an angioplasty balloon.”

U.S. Pat. No. 5,634,720, for a “Multi-purpose multi-parameter cardiac catheter,” discloses “A multi-lumen, multi-purpose cardiac catheter which incorporates optical filaments and an optical coupler for use with external apparatus for determining the oxygen concentration in the blood of a patient under critical care conditions, as well as incorporating therein a thermal element useable with a second external apparatus for measurement of continuous cardiac output.”

U.S. Pat. No. 5,435,308, for a “Multi-purpose multi-parameter cardiac catheter,” discloses “A multi-lumen, multi-purpose cardiac catheter which incorporates optical filaments and an optical coupler for use with external apparatus for determining the oxygen concentration in the blood of a patient under critical care conditions, as well as incorporating therein a heater coil useable with a second external apparatus for measurement of continuous cardiac output. The catheter also includes a thermistor and at least one injectate port for enabling the user to also conduct thermal dilution readings and obtain intermittent measurements of cardiac output. The combination of a thermal dilution catheter with a SVO2 catheter and a continuous cardiac output catheter gives the multi-purpose catheter above described substantial versatility as well as providing the user with a versatile cardiac catheter device which enables him to conduct multiple evaluations of disparate blood-related parameters which require the use of separate apparatus. Simply by switching from one external apparatus to the other, the user can obtain readings for different blood-related parameters useful in the treatment of the cardiac patient.”

U.S. Pat. No. 6,036,654, for a “Multi-lumen, multi-parameter catheter,” discloses “A multi-lumen catheter capable of measuring cardiac output continuously, mixed venous oxygen saturation as well as other hemodynamic parameters. The catheter is also capable of undertaking therapeutic operations such as drug infusion and cardiac pacing. The catheter includes optical fibers for coupling to an external oximeter, an injectate port and thermistor for bolus thermodilution measurements, a heating element for inputting a heat signal and for coupling to an external processor for continuously measuring cardiac output, and a distal lumen for measuring pressure, withdrawing blood, guidewire passage or drug infusion. In a preferred embodiment, the catheter includes a novel lumen configuration permitting an additional infusion lumen for either fast drug infusion or cardiac pacing.”

The disclosures of U.S. Pat. Nos. 6,236,879, 6,166,806, 5,973,779, 5,415,653, 4,991,590, 5,919,135, 6,026,316,6,052,613, 5,445,151, 6,134,003, 5,830,209, 6,024,738, 5,634,720, 5,435,308, and 6,036,654 are incorporated into this disclosure by reference.

Despite the advances in capabilities that are described in these numerous catheter devices, there remain shortcomings in the capabilities of these catheter devices, and in magnetic resonance imaging, and in the use of magnetic resonance imaging when these catheter devices are present in the body. As was previously described, there is a need to reduce the distance between the tissue to be imaged by MRI and the receive coil in the MRI unit. Because of the relatively large distance between the external receive coil in present MRI systems and the internal tissue of the patient, the signal-to-noise ratio is insufficient to provide a satisfactory image of certain tissues in many circumstances.

Many of these catheter devices are dangerous to the patient, because when such catheter devices are exposed to the MRI procedure, the metallic wires, tubing, structural supports, and other metallic leads therein are heated by the effect of the high frequency magnetic field. In addition, the functionality of these catheter devices is generally limited to a single purpose. It would be particularly beneficial to have a catheter device provided with multiple diagnostic features or capabilities in a single lead, and/or provided with diagnostic and therapeutic features in a single lead. In particular, it is highly desirable to incorporate an MRI coil into a catheter having additional diagnostic features or capabilities.

It is therefore an object of this invention to provide a small diameter MRI imaging coil that can be placed within the body, such as natural body openings or punctures through the skin, and to enable the coil to be positioned close to the tissues to be imaged, thereby providing significant improvement in morphological or spectral image quality due to the enhanced signal from the tissues and the increase in tissue magnetic resonance image signal-to-noise ratio that this closer proximity provides.

It is a further object of the present invention to combine with the present invention other diagnostic and therapeutic features and capabilities useful for the diagnosis and treatment of diseases.

### SUMMARY OF THE INVENTION

In accordance with the present invention, there is provided a catheter assembly comprising a cable assembly having a proximal end and a distal end, said cable assembly further comprising an outer tube, a first electronics assembly disposed within said distal end of said cable assembly, and a first fiber optic strand disposed within said tube and connected to said first electronic assembly; and a tip assembly connected to said distal end of said cable assembly further comprising a thin structural wall and a cavity formed within said thin structural wall, and a coil assembly disposed within said cavity, wherein said coil assembly is connected to said first electronics assembly.

### BRIEF DESCRIPTION OF THE DRAWINGS

The invention will be described by reference to the following drawings, in which like numerals refer to like elements, and in which:

FIG. 1 is a schematic of a cross section of a catheter bundle of optical strands,

FIG. 2 is a schematic of a cross section of a catheter bundle of optical and support strands,

FIG. 3 is a schematic of a cross section of a catheter bundle of optical, strands, tubes, and support strands, and

FIGS. 4-14 each schematically illustrate a numerous embodiments of a catheter cable and tip.

The present invention will be described in connection with a preferred embodiment, however, it will be understood that there is no intent to limit the invention to the embodiment described. On the contrary, the intent is to cover all alternatives, modifications, and equivalents as may be included within the spirit and scope of the invention as defined by the appended claims.

### DESCRIPTION OF THE PREFERRED EMBODIMENTS

For a general understanding of the present invention, reference is made to the drawings. In the drawings, like reference numerals have been used throughout to designate identical elements. In describing the present invention, the terms distal and proximal ends are used to describe the catheter embodiments disclosed herein. As used herein, the proximal end of a catheter is meant to describe the end thereof that is external to the body in which it is disposed. The distal end of a catheter is meant to describe the end thereof that is internal to the body in which it is disposed. The catheter terminates within such a body at the distal end of such catheter. FIGS. 4-14 of this disclosure depict distal ends of catheters of the present invention.

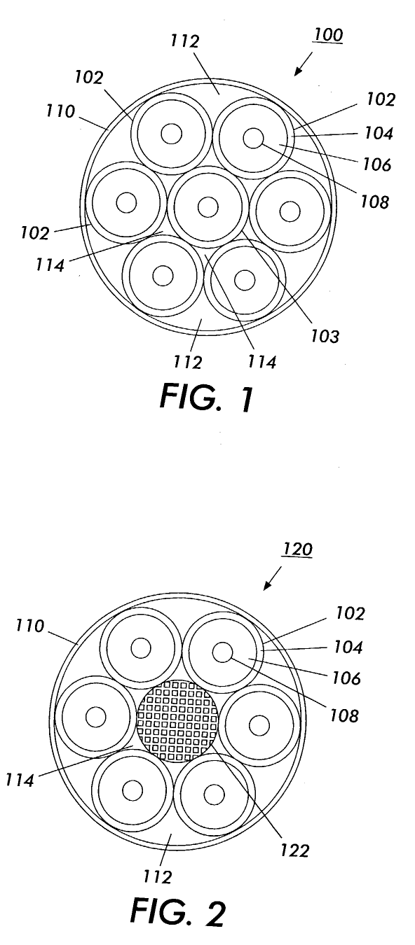

FIG. 1 is a cross-sectional view of a catheter cable assembly 100. Such catheter cable assembly 100 is typical of prior art optical cable assemblies. Reference may be had, e.g., to U.S. Pat. No. 4,784,461 (optical cable with improved strength), U.S. Pat. No. 6,259,843 (optical cable), U.S. Pat. No. 5,611,016 (dispersion balanced optical cable), U.S. Pat. No. 4,911,525 (optical communications cable), U.S. Pat. No. 4,798,443 (optical cable), U.S. Pat. No. 5,634,720 (multi-purpose multi-parameter cardiac catheter), and the like. The entire disclosure of each of these United States patents is hereby incorporated by reference into this specification.

Referring to FIG. 1, and in the preferred embodiment depicted therein, six fiber optic strands 102 are shown surrounding a central fiber optic strand 103. It is to be understood that the number of strands 102 in the assembly 100 of the catheter cable may be more or less than the number depicted. In one embodiment, from about 1 to about 10 such fiber optic strands 102 may be used.

Referring again to FIG. 1, it is preferred that each such fiber optics strand 102/103 be comprised of a core 108. This core 108 preferably consists of or consists essentially of silicon dioxide (silica), preferably of high purity. The core 108 generally has a symmetrical cross section, such as a circular cross section; and it usually has a diameter of from about. 1 to about 100 microns. In one embodiment, core 108 has a diameter of from about 2 to about 10 microns.

Cladding 106 preferably envelops the core 108. In the embodiment depicted, the cladding 106 has an outside diameter that is substantially larger than core 108, being at least about 1.1 times as large as the diameter of the core. In general, the cladding generally has a diameter of from about 5 to about 150 microns. In one embodiment, the optical cladding 106 has a thickness of approximately 60 micrometers and is itself preferably surrounded by a protective film 104. The protective film 104 preferably consists essentially of plastic material and, in one embodiment, has a thickness of approximately 1 micrometer. In the embodiment depicted in FIG. 1, six (6) of these fiber optic strands 102 comprising core 108 and cladding 106 are positioned around a central fiber optic strand 103.

In the embodiment depicted, the seven fiber optic strands 102/103 of FIG. 1 are surrounded by a protective layer comprising a sleeve or tube 110, which keeps the seven individual, strands 102/103 together. Such outer tubing 110 may be made from flexible material such as, e.g., plastic.

The regions 114 disposed between fiber optic strands 102/103 in one embodiment are preferably filled with additional material 114 to provide for increased structural strength of the overall assembly 100. In one embodiment, the additional material 114 is plastic material. In another embodiment, the additional material 114 is steel fiber or carbon fiber. In one embodiment, it is preferred that none of the materials within the cable assembly 100, and/or the cable assembly 120 (see FIG. 2) be electrically conductive.

In one embodiment, illustrated in FIG. 1, some or all of the outer regions 112 are filled with the same additional material(s) within spaces 114, and/or different additional material. Furthermore, some of these spaces 114/112 may be filled with additional material, whereas others are not.

Fewer or more interstrand regions 114/112 will exist depending on the total number of strands comprising the catheter cable assembly 100. The choice of material depends, in part, on the desired flexibility and strength of the catheter cable assembly 100.

FIG. 2 is a sectional view of an optical cable assembly 120 in which a central strand 122 is preferably comprised of, or consists essentially of, a single, solid material. In this embodiment, strand 122 may be used to give the catheter cable additional structural strength or flexibility. The additional, solid material 122 may be a plastic material, may be optically inert, and may preferably be electrically insulative.

It is preferred that the material 122 have low magnetic susceptibility. Thus, e.g., the material 122 can be made of glass-epoxy, quartz glass, or other material having a low magnetic susceptibility. As is known to those skilled in the art, magnetic susceptibility is measured by the ratio of the intensity of magnetization produced in a substance to the magnetizing force or intensity of field to which it is subjected.

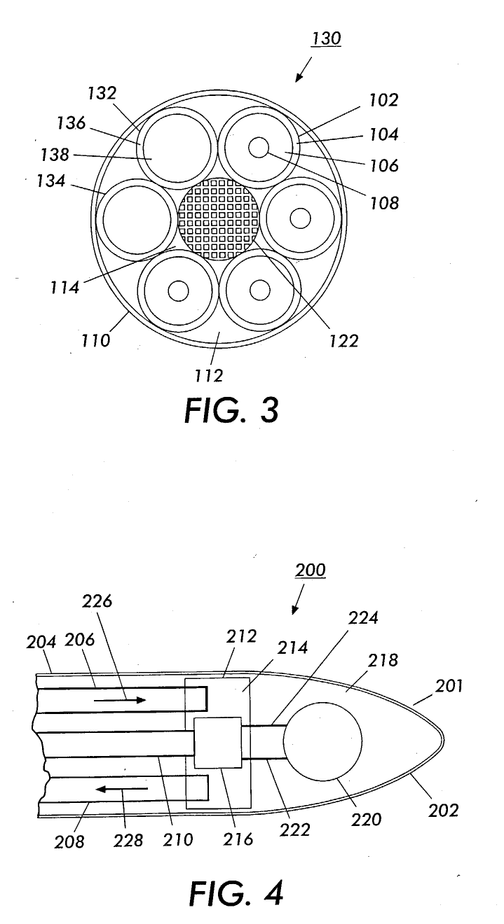

FIG. 3 depicts another embodiment of another cable strand assembly 130 in which two of the fiber optics strands 102 of FIG. 2 are replaced by lumens 132 and 134.

As will be apparent, these lumens may comprise and/or convey cooling fluid(s) or gas(es), heat exchange fluids or gases, and the like. The lumens 132 and/or 134 may be pressured. The lumens 132 and/or 134 may be partially evacuated.

In the preferred embodiment depicted in FIG. 3, lumens 132 and/or 134 preferably comprise a wall 136 of approximately 1 to 2 micrometers thick and an axial void 138 of approximately 125 micrometers in diameter.

FIG. 4 is a schematic representation of an assembly 200 comprised of a cable assembly 204 and a catheter tip assembly 201 connected to the cable assembly 204 at the distal end of the catheter (not shown).

As is known to those skilled in the art, a catheter is a tubular instrument adapted to allow passage of fluid, other material, or energy from or into a body cavity or blood vessel. As used herein, the term “catheter” refers to a tubular cable assembly connected to a tip comprised of a thin structural wall and a cavity enclosed therein, containing means for converting photonic energy to electrical energy, and vice versa.

Referring again to FIG. 4, catheter tip assembly 201 comprises a thin structural wall 202 containing a volume or cavity 218, within which a variety of small devices may be disposed. Catheter cable 204 preferably comprises at least two tubes 206 and 208 and a fiber optics strand 210. These tubes/strand 206/208/210 preferably pass into a sealed chamber 212. Disposed within the volume 214 of the chamber 212 is an electronic transducer assembly 216 connected to the fiber optics strand 210 and also connected to a coil assembly 220 situated outside the chamber 212, but within the tip volume 218. The connection of the electronic assembly 216 to the coil assembly 220 is preferably made by conductors 222 and 224.

The coil assembly 220 is preferably one or more pick-up coils and/or one or more transmit coils suitable for magnetic resonance imaging procedures. As is known to those skilled in the art, pickup coils are adapted to sense a signal or quantity. Reference may be had, e.g., to U.S. Pat. No. 4,691,164, which also describes coil 120 as being a “transmitter/receiver.” Reference also may be had, e.g. to U.S. Pat. Nos. 4,450,408, 6,278,277, 5,061,680, 5,158,932, and the like. The entire disclosure of each of these United States patents is hereby incorporated by reference into this specification.

Referring again to FIG. 4, the lumens 206 and 208 may be used, e.g. to cycle air through the chamber 212 to provide a cooling means for the electronics assembly 216. Such a flow may be made into and out of chamber 212, as is indicated by the flow direction arrows 226 and 228. Alternatively, a liquid may be cycled through the chamber 212 for the purposes of assisting and controlling the dissipation of heat generated by the electronics assembly 216.

In another embodiment (not shown), the catheter cable assembly 204 of FIG. 4 additionally contains a strand suitable for steering the catheter tip through the lumens of the body.

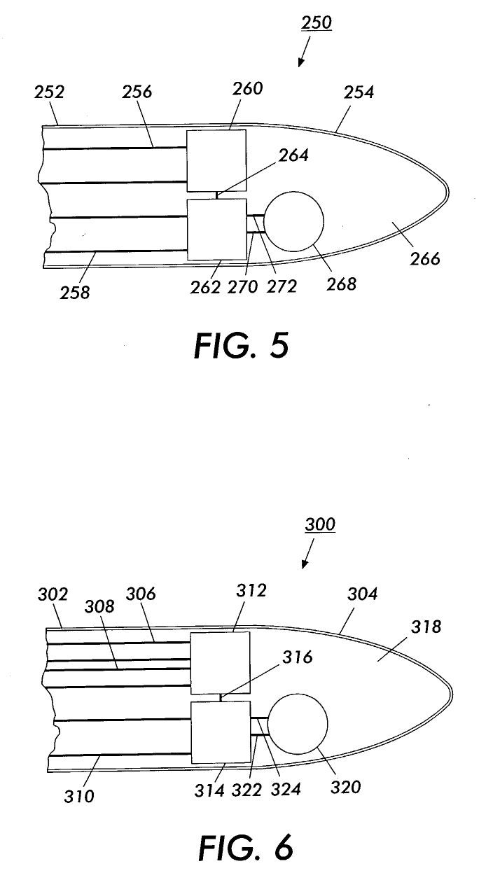

In another embodiment, illustrated in FIG. 5, one preferred assembly 250 of the distal end of the catheter cable assembly 252 and catheter tip 254 is illustrated. The cable assembly 252 consists of at least two strands 256 and 258. As will be apparent, the assembly 250 includes two separate electronic assemblies 260 and 262, and two strands 256 and 258.

Referring to FIG. 5, and in the preferred embodiment depicted therein, strand 256 is preferably connected to an electronic assembly 260 that preferably houses means for converting and storing energy conveyed to it through strand 256.

In one embodiment, depicted in FIG. 5, strand 256 is hollow tube or lumen filled with a gas (such as air), and/or a liquid, and/or a solid material(s). In this embodiment, the power assembly 260 may contain a piezoelectric crystal (not shown), one or more capacitors (not shown), one or more inductors (not shown), one or more resistors (not shown), and other electronic components, circuits, and assemblies (not shown).

Referring again to FIG. 5, and in one embodiment, the end of lumen 256 is connected to the piezoelectric crystal (not shown) in such a way as to oscillate the piezoelectric crystal as the pressure in the tube 256 is oscillated by an external means (not shown). By such a device, one can convert a pressure signal into an electrical signal, and vice versa. If one were to add photoelectric devices to this assembly, one would also be able to convert pressure signals to photonic signals, and vice versa.

As will be apparent, in the embodiment depicted in FIG. 5, hydraulic energy/signals may be converted to electrical energy/signals, and vice versa, by piezoelectric transducer assembly 260. Thus, in addition to conveying information photonically, the device 250 is also capable of transmitting information hydraulically.

In another embodiment, strand 256 is a fiber optics cable. Power assembly 260 may contain a photovoltaic cell (not shown) along with a capacitor (not shown). An external laser diode (not shown) may preferably send light through the strand 256 to the assembly 260 where it is converted to an electrical potential by a photovoltaic cell (not shown) which charges the capacitor.

In the embodiment illustrated in FIG. 5, strand 258 is preferably a fiber optics strand to be used for sending signals to the proximal end of the catheter cable 252. Strand 258 is preferably connected to an electronics assembly 262 at the distal end of the cable assembly 252. The electronics assembly 262 is preferably powered by the power assembly 260 through connection 264. The electronics assembly 262 preferably has means (not shown) for converting and sending signals received by one or more coils 268 through optics strand 258. The coils 268 are connected to the electronics assembly 262 via lines 270 and 272. In another embodiment, not shown, the coils 268 are telemetrically connected to the electronics assembly 262.

In one embodiment, not shown, several coils 268 are positioned at various angles to enhance the imaging ability of the catheter. As will be apparent, the angles at which radiation impacts an antenna often affect its receiving capabilities.

In another embodiment, not shown, the coil 268 may be rotated and/or translated into various angles and locations within the tip assembly by an actuator (not shown) controlled by electronics assembly 262.

FIG. 6 illustrates another embodiment of this invention comprising an assembly 300 comprised of a catheter cable assembly 302 and a distal end tip 304. The cable assembly 302 contains at least tubes 306 and 308 connected to a power assembly 312, and at least one fiber optics strand 310 connected to an electronics assembly 314. In this embodiment, liquid (or gas) may be cycled through the power assembly 312 which is so constructed, in one embodiment, as to convert the motion of the fluid through assembly 312 or to convert the contents of the liquid (or gas) into electrical energy suitable for running the electronics in assembly 314. The liquid or gas, e.g. may contain electrolytes, and assembly 312 may be so constructed as to comprise a battery. The power assembly 312 is connected to the electronics assembly 314 via line 316.

In one embodiment, the electronics assembly 314 is connected to the fiber optics strand 310 and is used to convey signals obtained from coils 320, which are connected to the electronics assembly 314 via lines 322 and 324, through the optics strand 310. Additionally, strand 310 may be used to send signals from the external proximal end (not shown) of the cable assembly 302 to the electronics assembly 314.

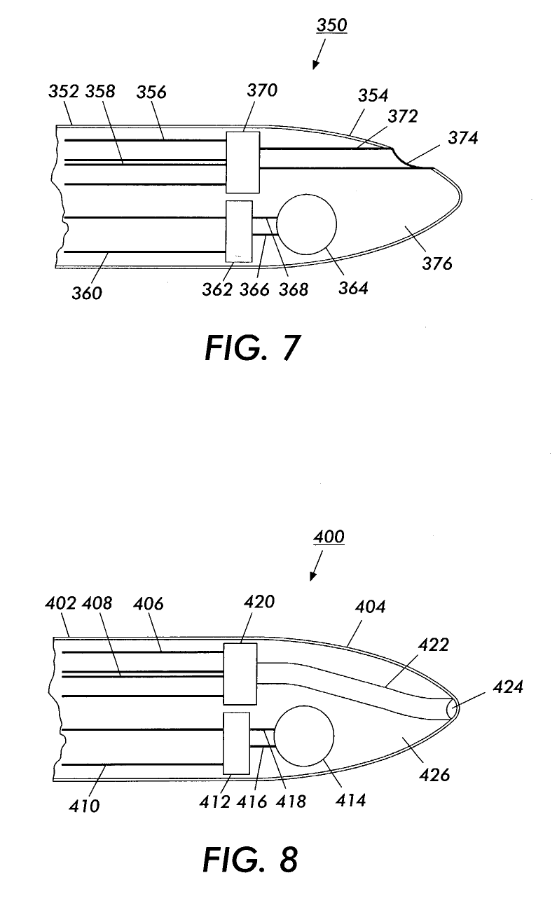

In another embodiment depicted in FIG. 7, an assembly 350 is shown comprising a catheter cable assembly 352 and a catheter tip assembly 354. The catheter cable assembly comprises at least 3 strands, 356, 358, 360.

In this embodiment, strands 356 and 358 are connected to a subassembly 370. Subassembly 370 is connected to a syringe needle 372 that has an open orifice 374 in the tip 354. In one embodiment of the configuration depicted in FIG. 7, strand 356 is a hollow tube and strand 358 is a fiber optic. Subassembly 370 may consist of a reservoir (not shown) and electronic means (not shown) for controlling the release of the reservoir contents through the needle 372. Strand 356 is then used to fill the reservoir with the desired solution, e.g. an MRI contrast agent or drug, or topical ointments, etc. Strand 358 may be used to communicate externally with the electronics of subassembly 370 to signal when the solution stored in the reservoir is to be released.

In another embodiment of FIG. 7, not shown, the needle 372 is used to obtain fluid samples from the body. In this embodiment, tube strand 356 is used to provide a vacuum pressure suitable for drawing the bodily fluid through the needle 372. Subassembly 370 is so constructed as to provide means for controlling the drawing of a fluid through the needle 372. Subassembly 370 may also contain medical analyses means (not shown) suitable, e.g. for detecting glucose levels in blood, for detecting toxins in the blood, for determining the pH level of the sampled fluid, etc. Subassembly 370 also preferably has means (not shown) for sending data pertaining to the results of such analysis through the fiber optics strand 358 to an external monitor or physician (not shown). Additionally, strand 358 may be used to send command signals from an outside physician to the subassembly 370 to control the drawing of fluid and to direct the analysis of said drawn fluid.

Referring again to FIG. 7, the electronics assembly 362 is preferably connected to the fiber optics strand 360 and is used to convey signals obtained from one or more coils 364, which are connected to the electronics assembly 362 via lines 366 and 368, through the optics strand 360. Additionally, fiber optics stand 360 may be used to send signals from the external proximal end (not shown) to the cable assembly 352 to the electronics assembly 362.

FIG. 8 depicts another embodiment of an assembly 400 comprised of a catheter cable assembly 402 and a tip assembly 404. The catheter cable assembly 402 comprises of at least 3 strands 406, 408, 410 that, in this embodiment, are all preferably fiber optics strands. In this embodiment, strands 406 and 408 are connected to optical electronics assembly 420. Also connected to optical electronics assembly 420 is an optics conduit assembly 422 that is connected to a lens assembly 424 built into the outer surface of the tip assembly 404. The optical electronics assembly 420, optics conduit assembly 422 and lens assembly 424 may comprise the components of an optical biopsy assembly or may provide means for performing Optical Coherent Tomography. In these cases, the optics strand 406 may convey the light to be used for the optical biopsy procedures, while optics strand 408 is used by the electronics assembly 420 to convey the biopsy information back to the physician or external monitoring device (not shown). Additionally, optical electronics assembly 420, optics conduit assembly 422, and lens assembly 424 may be used for laser ablation at the tip site. Laser light may be generated by a laser diode built into optical electronics assembly 420, or may be obtained from an external source through strand 406. In another embodiment, the functionality of strand 406, optical electronics assembly 420, optics conduit assembly 422 and lens assembly 424 is switched between performing, e.g., optical coherent tomography and laser ablation. Such functional switching may be controlled externally by communication between an external physician and the optical electronics assembly 420 Via fiber optic strand 408.

In another embodiment, and continuing to refer to FIG. 8, the electronics assembly 420 and optics assemblies 422 and 424 are utilized to provide video images of the external tip environment (not shown) through the optical strand 406 to the proximal end of the catheter.

Continuing to refer to FIG. 8, the electronics assembly 412 is preferably connected to the fiber optics strand 410 and is used to convey signals obtained from coils 414, which are connected to the electronics assembly 412 via lines 416 and 418, through the optics strand 410.

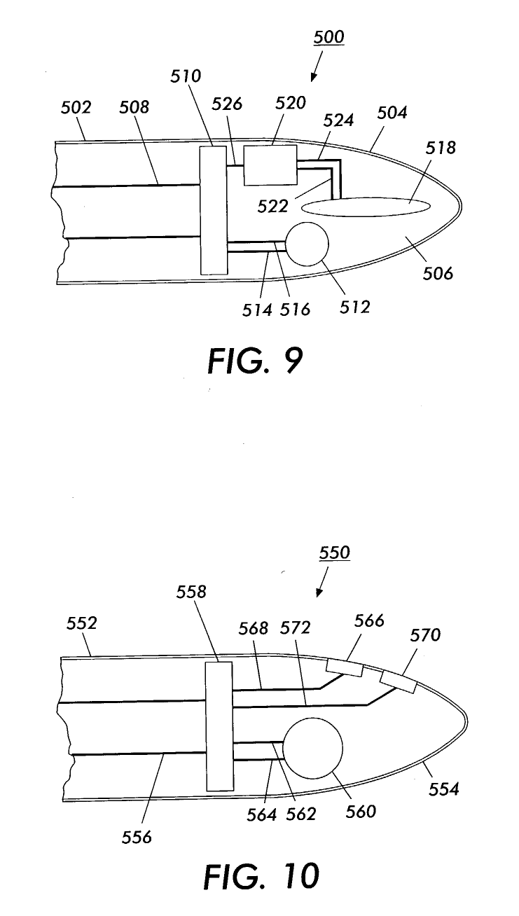

FIG. 9 depicts another embodiment of an assembly 500 comprising a cable assembly 502 and a tip assembly 504. In this embodiment, at least one fiber optic strand 508 is disposed within the catheter cable assembly 502. It is connected, within a tip cavity region 506, to an electronics assembly 510. The electronics assembly is connected to at least one coil 512 by means of lines 514 and 516. Signals from the coils 512 are converted into light signals by the electronics assembly 510 and sent out through the fiber optic strand 508. The power to run the electronics assembly 510 is preferably provided by a power electronics assembly 520, which is connected to at least one coil 518 via lines 522 and 524. In magnetic resonance imaging (MRI) technology, external radio frequency electromagnetic waves are applied to the a body in order to excite protons in the nuclei of the body's atoms. The coils 518 and power electronics 520 are so designed as to resonate at the externally applied radio frequency wave frequency. In this way, energy may be delivered, and possibly stored in capacitors (not shown) within power electronics assembly 520. The electrical power is provided to the electronics assembly 510 via line 526.

FIG. 10 depicts another embodiment of an assembly 550 comprising a cable assembly 552 and a tip assembly 554. In this embodiment, the cable assembly 552 comprises of at least one optics strand 556 connected to an electronics assembly 558. The electronics assembly 558 is connected to at least one pickup coil 560 via lines 562, 564. The electronics assembly 558 converts the signals picked up by the coils into light signals suitable for transmission through the fiber optics strand 556 and generates and transmits such signals. Additionally, other sensors 566, and electromagnetic emitters 570 are connected to the electronics assembly 558 via lines 568 and 572. Sensors 566 and emitters 570 are also connected to, and may protrude through, the tip 554. Sensors 566 may be used, e.g., to sense the temperature, blood pressure, blood flow rate, etc. within a body. Emitters 570 may be used, e.g. to emit millimeter electromagnetic energy, or heat, or other energy. The electronic assembly 558 collects sensed data from the sensors and converts the data into light signals suitable for transmission through the fiber optics strand 556. Electronics assembly 558 also controls and coordinates which datum from which sensor and/or coil is to be transmitted through fiber optics strand 556 at ay given time.

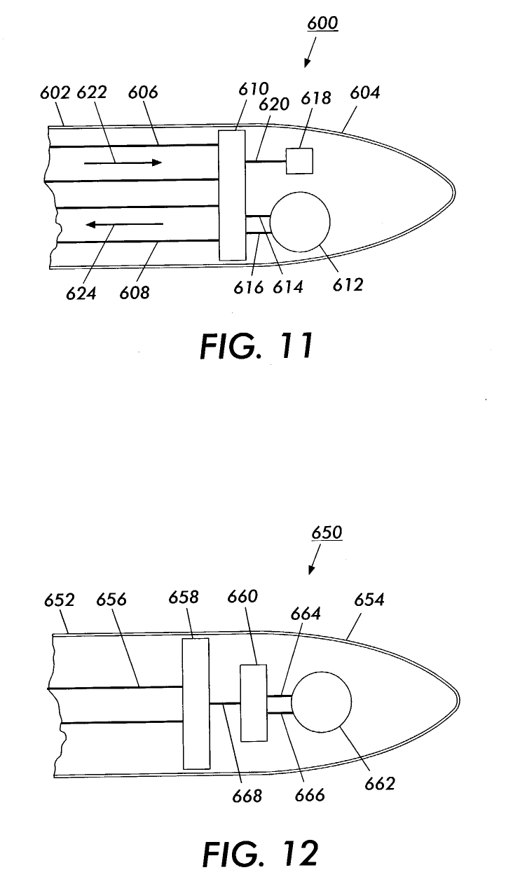

FIG. 11 depicts another embodiment of an assembly 600 comprising a cable assembly 602 and a tip assembly 604. The cable assembly 602 comprises at least 2 fiber optic strands 606, 608 connected to an electronics assembly 610. The electronics assembly 610 is connected to at least one sensing device, including, but not limited to, a pickup coil 612. Other, optional, sensing devices are labeled as 618. The electronics assembly 604 is connected to the pickup coil 612 via lines 614, 616. The other sensing devices are connected to the electronics assembly 610 via line 620.

In this embodiment, laser light, or other suitable light, is sent from an external source (not shown) through fiber optics strand 606 as indicated by arrow 622 to the electronics assembly 610. The electronics assembly modifies the light in a predetermined way to encode the signals from the coil 612 and/or the sensing devices 618 and then channels the light through the fiber optics strand 608, as indicated by arrow 624. In this way, a source for generating light is not required at the electronics assembly 610. One method for encoding a signal is to construct electronics assembly 610 with optical components suitable for causing phase shifts in the light 622 based on signals from the coil 612 or other sensing devices 618. Then, by externally comparing the phase between the light sent in 622 with that of the light sent out 624, a means for transmitting sensed data is realized. Other means for altering the incoming light 622 before channeling it out as 624 may be utilized. Using such techniques reduces the power requirements of the electronics assembly 610 since, in these embodiments, electronics assembly 610 does not need a light source. Also, this provides a way to utilize light sources that might not otherwise be applicable if it were required to be part of the electronics assembly 610 because of size constraints, power requirements, or heating problems. Using an external light source as described here eliminates these constraints.

In another embodiment depicted in FIG. 12, a distal end catheter assembly 650 comprises a catheter cable assembly 652 and a tip assembly 654 suitable for performing radio frequency ablation within a body. Other frequencies of electromagnetic energy outside of the radio frequency range may also be utilized. The catheter cable assembly comprises at least one optical strand 656 connected to an electronic assembly 658 which contains means for converting the optical energy sent from the external proximal end of the catheter (not shown) to the electronic assembly 658 at the distal end of the catheter. Such means for converting the optical energy to electrical energy may be, e.g., a photovoltaic cell. Electronic assembly 658 may also be comprised of other electronic components as well. The electronic assembly 658 is connected to an radio frequency signal generator 660 via line 668. The radio frequency signal generator 660 is connected to one or more coils 662 suitable for performing, e.g. radio frequency ablation, via lines 664, 666.

In another embodiment (not shown) the radio frequency generator of the embodiment shown in FIG. 12 is removed. In this case, the optical energy sent to the electronic assembly 658 of FIG. 12 is pulsed at the desired radio frequency. Other frequencies outside of the radio frequency range may also be utilized. The electronics assembly 658 of FIG. 12 is correspondingly modified to connect directly to the coils 662 of FIG. 12. In this way, the amount of electronics, power requirements, heat generation and possibly other constraints in the design of the catheter tip may be reduced.

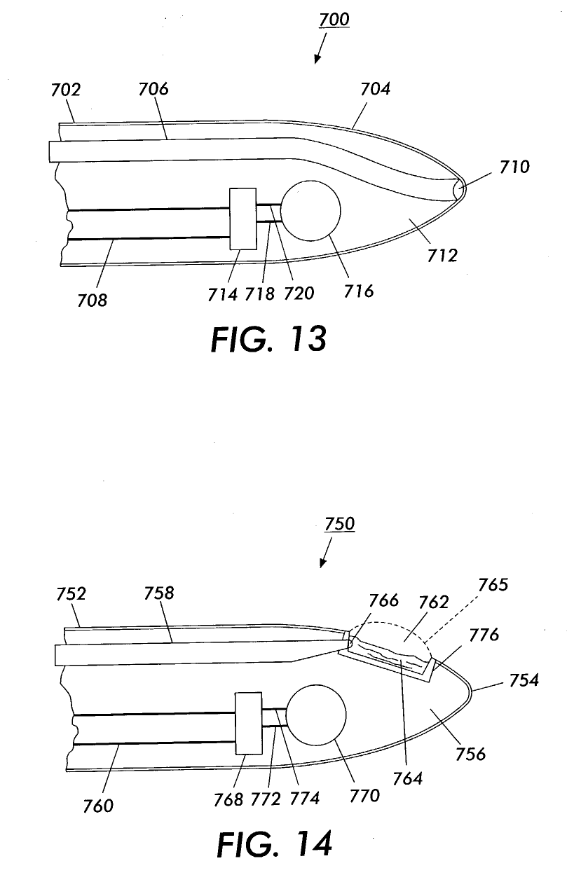

FIG. 13 depicts another embodiment of an assembly 700 comprised of a catheter cable assembly 702 and a tip assembly 704. The catheter cable assembly 702 comprises 2 strands 706, 708 that, in this embodiment, are all preferably fiber optic strands. In this embodiment, strand 706 passes through the tip area 712 and connects to a lens assembly 710. Thus, electromagnetic energy (such as, e.g., optical energy, microwave energy, millimeter wave energy, and the like) from a source (not shown) at the remote proximal end of the catheter cable 702 may be directly applied to the tip 704 and to the external environment disposed beyond it.

In one embodiment, the electromagnetic energy conveyed through 706 is outside of the visible electromagnetic spectrum, includes the near infrared, and/or infrared and/or ultraviolet, and/or other ranges of the electromagnetic spectrum. In another embodiment, and continuing to refer to FIG. 13, the optical energy passed through the strand 706 and out through the lens assembly 710 is a laser light adapted to apply heat to the environment proximate to the tip. In another embodiment, the laser energy may be utilized for cauterization.

Continuing to refer to FIG. 13, the electronics assembly 714 is preferably connected to the fiber optics strand 708 and is preferably used to convey through optics strand 708 the signals obtained from coils 716 that are connected to the electronics assembly 714 via lines 718 and 720.

FIG. 14 depicts another embodiment of the invention, illustrating an assembly 750 comprised of a catheter cable assembly 752 and a tip assembly 754: The catheter cable assembly 752 comprises two strands 758, 760. Strand 758 is preferably a hollow lumen or tube suitable for transporting a gas or a liquid. Strand 760 is preferably a fiber optic strand. In this embodiment, strand 758 connects to one or more inflatable bladders 764 disposed within the tip volume 756. The connection of the tube 758 to the bladder(s) is accomplished via connection assembly 766. The bladder is further enclosed within a chamber 776 within the tip volume 756 which provides the necessary constraints on the bladder 764 such that when a gas or liquid is pumped into the bladder 764, said bladder 764 can not extend into the tip volume 756. The bladder 764 is so disposed as to be able to expand out of the tip 754 through orifice 762 of tip 754. In this way, the catheter tip 754 may be stabilized within the body environment (not shown) to which said tip is introduced. Applying a partial vacuum to the tube 758 retracts the bladder 764.

Continuing to refer to FIG. 14, the electronics assembly 768 is preferably connected to the fiber optics strand 760 and is used to convey signals obtained from one or more coils 770, which are connected to the electronics assembly 768 via lines 772 and 774, through the optics strand 760. The electronic assembly 768 may contain means for decoupling the coils 770 with respect to the externally applied (not shown) magnetic resonance imaging radio frequency and/or gradient magnetic field oscillations. Additionally, electronics assembly 768 may contain means for converting the signals picked up by the coils 770 into digital signals or analog signals suitable for transmission through the fiber optics strand 760. Multiplexing of signals may also be used to transmit and/or receive signals through fiber optics strand 760.

In another embodiment (not shown), one or more bladders are disposed along the cable assembly 752 of FIG. 14, rather than or in addition to the bladders in the tip 754 of FIG. 14.

In another embodiment (not shown), extendable and retractable wires are used to increase the stability of the catheter tip.

It is, therefore, apparent that there has been provided, in accordance with the present invention, a catheter assembly that is compatible with and that may be subjected to a magnetic resonance imaging process without adverse effects on the assembly, or the patient within whom it is disposed. While this invention has been described in conjunction with preferred embodiments thereof, it is evident that many alternatives, modifications, and variations will be apparent to those skilled in the art, and that changes can be made in the apparatus, in the ingredients and their proportions, and in the sequence of combinations and process steps, as well as in other aspects of the invention discussed herein. Accordingly, it is intended to embrace all such alternatives, modifications and variations that fall within the spirit and broad scope of the appended claims.

I claim:

1. A catheter assembly comprising
(a) a cable assembly having a proximal end and a distal end, said cable assembly further comprising an outer tube, a first electronics assembly disposed within said distal end of said cable assembly, and a first fiber optic strand disposed within said outer tube and connected to said first electronic assembly, and
(b) a tip assembly connected to said distal end of said cable assembly further comprising a thin structural wall and a cavity formed within said thin structural wall; and a coil assembly disposed within said cavity, wherein said coil assembly is connected to said first electronics assembly.

2. The catheter assembly as recited in claim 1, further comprising a first tube and a second tube disposed within said outer tube of said cable assembly and passing into a sealed chamber disposed within said distal end of said cable assembly, wherein said fiber optic, strand passes into said sealed chamber, and said electronics assembly is disposed within said sealed chamber.

3. The catheter assembly as recited in claim 2, wherein one of said first tube and said second tube contains a gas flowing into said sealed chamber, and the other of said first tube and said second tube contains said gas flowing out of said sealed chamber.

4. The catheter assembly as recited in claim 2, wherein one of said first tube and said second tube contains a liquid flowing into said sealed chamber, and the other of said first tube and said second tube contains said liquid flowing out of said sealed chamber.

5. The catheter assembly as recited in claim 1, further comprising a strand disposed within said cable assembly, said strand connected to a second electronics assembly comprising means for converting and storing energy conveyed thereto through said strand.

6. The catheter assembly as recited in claim 1, further comprising a lumen disposed within said cable assembly, said lumen connected to a second electronics assembly disposed within said distal end of said cable assembly and comprising a piezoelectric crystal.

7. The catheter assembly as recited in claim 1, further comprising a second fiber optic strand disposed within said outer tube of said cable assembly, said second fiber optic strand connected to a second electronics assembly disposed within said distal end of said cable assembly.

8. The catheter assembly as recited in claim 7, wherein said second electronics assembly further comprises a photovoltaic cell.

9. The catheter assembly as recited in claim 7, wherein said second electronics assembly is a power assembly, and said first electronics assembly is electrically connected and powered by said second electronics assembly.

10. The catheter assembly as recited in claim 1, further comprising a first tube, and a second tube disposed within said outer tube of said cable assembly and connected to a power assembly disposed within said distal end of said cable assembly, wherein a portion of the energy of a fluid flowing from said first tube into said power assembly and out through said second tube is converted to electrical energy by said power assembly.

11. The catheter assembly as recited in claim 10, wherein said said first electronics assembly is electrically connected and powered by said power assembly.

12. The catheter assembly as recited in claim 1, further comprising a tube disposed within said outer tube of said cable assembly and passing into a subassembly disposed within said distal end of said cable assembly comprising a having an open orifice disposed through said thin structural wall of said tip assembly.

13. The catheter assembly as recited in claim 12 further comprising a second fiber optic strand disposed within said outer tube of said cable assembly and passing into said subassembly, wherein said subassembly further comprises a second electronics assembly.

14. The catheter assembly as recited in claim 13, wherein said subassembly further comprises a fluid reservoir.

15. The catheter assembly as recited in claim 14, wherein said second electronics assembly further comprises means for controlling the release of fluid from said reservoir through said needle.

16. The catheter assembly as recited in claim 14, wherein said second electronics assembly further comprises means for controlling the drawing of fluid from said needle inwardly to said reservoir.

17. The catheter assembly as recited in claim 16, further comprising medical analyses means for analyzing fluids drawn in through said needle.

18. The catheter assembly as recited in claim 1, wherein said cable assembly further comprises a second fiber optic strand and a third fiber optic strand disposed within said outer tube of said cable assembly, said second fiber optic strand and said third fiber optics strand connected to an optical electronics assembly disposed within said distal end of said cable assembly; and said tip assembly further comprises a lens assembly disposed in the outer surface of said tip assembly, and an optics conduit assembly connected to said lens assembly and said optical electronics assembly.

19. The catheter assembly as recited in claim 18, wherein said optical electronics assembly further comprises a laser diode providing laser light through said optics conduit assembly connected to said lens assembly.

20. The catheter assembly as recited in claim 1, wherein said tip assembly further comprises a receiving coil for receiving radio frequency electromagnetic waves connected to a power assembly, and wherein said power assembly is electrically connected to said electronics assembly.

21. The catheter assembly as recited in claim 1, wherein said tip assembly further comprises an electromagnetic sensor disposed in the outer surface thereof and connected to said electronics assembly.

22. The catheter assembly as recited in claim 1, wherein said tip assembly further comprises an electromagnetic emitter disposed in the outer surface thereof and connected to said electronics assembly.

23. The catheter assembly as recited in claim 1, further comprising a radio frequency generator disposed within said cavity formed within said thin structural wall of said tip assembly, wherein said radio frequency generator is connected to said electronics assembly and to said coil assembly.

24. The catheter assembly as recited in claim 1, further comprising a second fiber optic strand disposed within said outer tube of said cable assembly, said second fiber optic strand extending through said tip assembly to a lens assembly disposed in the outer surface of said tip assembly.

25. The catheter assembly as recited in claim 1, further comprising a lumen disposed within said cable assembly, an inflatable bladder enclosed within a chamber in said tip assembly, wherein said inflatable bladder is disposed along an orifice in said thin structural wall of said tip assembly, and wherein said lumen is connected by a connection assembly to said inflatable bladder.
